(19) United States
(12) Patent Application Publication
(10) Pub. No.: US 2011/0105848 A1
(43) Pub. Date: May 5, 2011

(54) LAPAROSCOPIC TISSUE RETRACTOR

(76) Inventors: Niv Sadovsky, Tel Aviv (IL); Haim Eliash, Zichron Yaakov (IL); Tzur Di-Cori, Modi'in (IL)

(21) Appl. No.: 12/734,043

(22) PCT Filed: Oct. 7, 2008
(86) PCT No.: PCT/IB2008/054110

§ 371 Date: Dec. 21, 2010

(30) Foreign Application Priority Data
Oct. 7, 2007 (US) 60978125

(51) Int. Cl. A61B 1/32

(52) U.S. Cl. 600/204

(57) ABSTRACT

The present invention is primarily directed to a surgical retractor suitable for laparoscopic insertion, comprising an elongate shaft having two or more arms at its distal end, and a mechanism for controlling the mutual separation of said arms located at its proximal end, wherein a membrane is attached to said two or more arms, such that upon mutual separation of said arms, said membrane forms a non-planar surface suitable for use as a barrier for retracting or holding tissues or organs.

### FIELD OF THE INVENTION

The present invention relates to a laparoscopic surgical retraction instrument that may be used to retain and/or move internal organs in a surgical field during minimally invasive or endoscopic surgery. The present invention also provides a laparoscopic port suitable for use with said retraction instrument.

### BACKGROUND OF THE INVENTION

Endoscopic surgery involves indirect visualization of the operative field with a small camera or optic fibers. Endoscopic surgery is generally done by way of multiple small incisions through which the camera and surgical instruments are inserted. The instruments perform their functions inside the body but are operated by means of handles that extend outside the body. Examples of commonly-performed endoscopic surgical operations include endoscopic appendectomy and laparoscopic cholecystectomy. Endoscopic surgery can also be performed through existing, natural orifices, for example, prostate surgery (via the urethra) and gastrointestinal surgery (via the oral or anal orifices).

During such procedures, the surgeon is required to expose and handle delicate tissues deep within the body cavities. This requires the creation and delineation of a surgical window, or workspace, through which the procedure can be performed. Ideally, the surgical window should be optimized such that it is wide enough to view and work within the treatment area, while reducing damage to the surrounding tissues to the absolute minimum.

In endoscopic surgery, the surgeon uses a variety of different instruments in order to manipulate the tissues within the surgical window. Common instruments of this type include graspers and clamps. In addition, most laparoscopic procedures require the use of retractors in order to move and retain tissues and organs (e.g. the body wall, the intestines and other large organs) that may otherwise obscure the operative site. Such retractor devices are generally inserted into the body cavity in a collapsed conformation through an additional incision, and are then expanded within the body and either held by an assistant or fixed to a stable object outside the body, most commonly to the operating table. The use of a grasper device or of currently used endoscopic retractors requires an additional entry aperture in the abdominal wall and an additional trocar. The grasper is operated by a surgeon/assistant, thus focusing some of his attention and occupying at least one of his hands with a relatively unimportant task. Furthermore, holding an organ with the grasper for a considerable period of time and moving it around while exerting all the force on one point, may harm the tissue especially delicate tissues, such as blood vessels and nerves. Therefore, from time to time, at the surgeon's discretion, the grasper should be released for short periods of time or the blades of the retractor should be shifted to relieve the pressure on any one point. Additionally, if the surgeon is involved in a long and delicate portion of the operation, the grasper may not be released for a significant amount of time. In addition, currently used retractors obstruct the view and require frequent repositioning.

Endoscopic surgery imposes many additional and specific difficulties on the surgeon, including the need to master the use of unfamiliar, non-intuitive tools, performance of the procedure at one place while looking in another direction and lack of immediate manual feedback. An additional important obstacle is the tendency of certain tissues or organs to invade the surgical workspace and occlude the visual field, such as small bowel loops descending into the pelvis while performing pelvic surgery and healthy tissues covering a tumor during resection.

An important case study that illustrates the importance of the surgical access window is laparoscopic cholecystectomy, which carries specific procedure-related complications due to the unique endoscopic procedure (A. Shamiyeh, W. Wayand: Laparoscopic cholecystectomy: early and late complications and their treatment. Langenbecks Arch Surg (2004) 389:164-171.). Over 600,000 patients are operated annually for gallstone disease in the United States, and over 75% of the operations are performed by laparoscopy. However, with the widespread acceptance of this operation all over the world, the spectrum of complications in gallstone surgery has changed and encompasses the full range of problems commonly encountered in minimally-invasive surgery. In the context of gallstone surgery, these complications include bleeding from vascular injury, biliary leaks, spillage of gallstones, bile duct injuries and bowel injuries. The most serious complication is transection of the bile duct, which is usually a consequence of inadequate exposure of the surgical site (E. M. Targarona, C. Marco, C. Balague', J. Rodriguez, E. Cugat, C. Hoyuela, E. Veloso, M. Trias. How, when, and why bile duct injury occurs. Surg Endosc (1998) 12: 322-326.)

The incidence of major vascular injuries in laparoscopy (including injuries of the aorta, iliac vessels, vena cava, inferior mesenteric arteries and lumbar arteries) is 0.07%-0.4%. The incidence of minor vascular injuries (e.g. to the branches of the epigastric vessels, mesenteric and omental vessels) is 0.1%-1.2% and the mortality rate is 0.05%-0.2% (Catarci M, Carlini M, Gentileschi P, Santoro E (2001).

Bowel injuries may occur during the insertion of the trocars and during dissection of the tissues. They often remain undetected during the operation and have been reported to occur in up to 0.87% of cases (Bishoff J T, Allaf M E, Kirkels W, Moore R G, Kavoussi L R, Schroder F (1999) Laparoscopic bowel injury: incidence and clinical presentation. J Urol 161:887-890.)

As mentioned hereinabove, the problem of surgical site access is currently addressed by using two classes of instruments: graspers that hold the organs in place and retractors that prevent tissues from interfering with the surgical window. The use of both of these technologies has significant limitations, including:
Difficult access despite their use, limiting view in the area where the retractor is used
Uncomfortable procedure and working space, since these tools are robust and space occupying
A surgical assistant is usually required to use the tools for tissue retraction (the grasper is operated by a surgeon, focusing his attention and occupying at least one of his hands with a relatively unimportant task), thus increasing the work-force needed for the procedure and the cost of the procedure
Difficult visualization, since these tools are not transparent, and block the view of the retracted tissues
The force used for retraction is non-controlled

During laparoscopic surgery, two additional common maneuvers used to address this problem are the positioning of the patient in the Trendelenburg position and inflation of the peritoneal cavity with gas. While these help to maintain a better surgical window, they may compromise the patient's cardiac or respiratory function, and are associated with an increased incidence of medical complications. This is especially significant when operating on elderly or ill patients who already have compromised cardiorespiratory function. In certain cases the problem might be so severe as to have the anesthesiologist forbid use of these maneuvers.

The Trendelenburg position involves inclination of the patient's body with his or her head down and legs up. When positioned in this manner, the patient's small bowel tends to glide away from his pelvis, enabling an easier approach to that area. However, at the same time the small bowel pushes the diaphragm upwards, thus interfering with the respiratory and circulatory processes.

Gas insufflation of the peritoneal cavity is necessary for creation of a working space. It is most commonly performed using CO₂ at a pressure of up to 14 mmHg. Lower pressures are sufficient for mobilization of the internal organs, but at least 12 mmHg are necessary for good visualization. Clinical studies and experience suggest that higher intraabdominal pressures are associated with increased cardiac, respiratory, hepatic, and surgical complications, and it was suggested that they be kept as low as possible, and certainly not higher than 12 mmHg.

In summary, good exposure of the surgical space is essential for clinical success of the procedure, especially in endoscopic procedures. Current surgical tools do not meet the need for optimizing the surgical window, and there is thus a significant need for devices that can optimize the surgical exposure by retraction of the surrounding tissues, eliminating the need for Trendelenburg positioning and high-pressure gas insufflations.

It is therefore an object of the present invention to provide a laparoscopic retraction device that may be used in a hands-free manner.

It is another object of the present invention to provide a laparoscopic retractor of the aforementioned kind which may additionally be used to manually move and grasp tissues and organs at the operative site.

Other objects and advantages of the invention will become apparent as the description proceeds.

### SUMMARY OF THE INVENTION

The present invention is primarily directed to a surgical retractor that is suitable for use in laparoscopic or minimally-invasive surgery, wherein said retractor comprises an elongate shaft ending in two or more distal arms, to which are attached a barrier membrane or mesh. The device of the present invention is characterized, firstly, by the curved surface presented by the barrier membrane or mesh when the distal arms are caused to mutually separate, and secondly, by the ability to precisely control the degree of mutual separation (“opening”) of the distal arms, such that the degree of curvature of the barrier surface may be correspondingly controlled or altered. One key advantage of the laparoscopic retractor of the present invention that arises directly from its unique structure is the fact that it may be used to effectively retract or hold back tissues or organs at any degree of distal arm opening. The functional consequence of this feature is that the operator may actively alter the shape of the barrier in order to conform to the shape of the organs or other structures that require retraction or grasping.

Thus in one aspect, the present invention provides a surgical retractor suitable for laparoscopic insertion, comprising
an elongate shaft having two or more arms at its distal end, and a mechanism for controlling the mutual separation of said arms located at its proximal end,
wherein a membrane is attached to said two or more arms, such that upon mutual separation of said arms, said membrane forms a non-planar surface suitable for use as a barrier for retracting or holding tissues or organs.

In one preferred embodiment of the device of the invention the non-planar barrier formed upon mutual separation of the distal arms is characterized by being curved along at least two separate axes of symmetry, and thus may be likened to the concave surface of the human hand (i.e. the palm) when the fingers are flexed.

It is to be emphasized that the term “membrane” is used herein to include within its scope non-porous membranes, porous membranes and meshes.

Preferably, each of the distal arms of the aforementioned surgical retractor is curved in one or more planes.

In one particularly preferred embodiment, the retractor comprises no more than two arms at its distal end.

The aforementioned elongate shaft may be solid or hollow, and may consist of a single elongate element or a plurality of such elements. In one preferred embodiment, however, the elongate shaft comprises a hollow tube through which pass two connecting elements (in the form of wires, rounded rods or flat elongated plates), each of which connects one of the distal arms with the proximally-located mechanism for controlling the mutual separation of said distal arms.

While there are several different types of proximally-located mechanisms for controlling the mutual separation of said distal arms (as will be described in more detail hereinbelow), in a particularly preferred embodiment, said mechanism comprises a pair of scissor handles, wherein one connecting element is attached to a fixed scissor handle, and wherein the second connecting element is fixed to a movable scissor handle, wherein said movable handle is capable of being caused to move between a plurality of defined positions, and is further capable of becoming immobilized into each of said defined positions. Preferably the movement of the movable handle in relation to the fixed handle is mediated by a ratchet mechanism.

In one highly preferred embodiment, the aforementioned is a polymeric mesh. In one preferred embodiment, this mesh is formed from polyethylene. Other suitable mesh materials will be described hereinbelow.

In another particularly preferred embodiment, the elongate shaft of the retractor device is enclosed by an expandable sleeve or sheath. Preferably (but not exclusively), said sleeve is a two-layer sheath formed from silicone or a similar material. Said sleeve is provided with an inflation tube such that it may be expanded in a balloon-like manner upon introduction of an expansion medium such as water, saline or air.

The aforementioned surgical retractor may also further comprise means capable of anchoring said retractor to an immobile structure (such as the operating table, surgical drapes etc.) Preferably, these means are provided in the form of one or more straps attached at their medial ends to the proximal part of said retractor, and wherein each of said straps further comprises a clip attached to the free lateral end thereof.

In another aspect, the present invention also provides a flexible laparoscopic port suitable for use in inserting a surgical retractor according to any one of the above claims into a body cavity, wherein said port comprises:
a flexible, elongate hollow tube suitable for passage of a surgical instrument therethrough;
an upper housing surrounding the proximal end of said tube, wherein said housing comprises one or more gas-tight seals;
a retention structure surrounding a portion of the distal part of said tube; and
a hollow distal tip contained within the distal end of said tube, wherein said tip comprises a circumferential step within its internal lumen.

Although various combinations of gas-tight seals may be used to construct the flexible port, in a preferred embodiment the upper housing comprises both:
an upper seal comprising having a central aperture that is capable of expanding in diameter in order to provide a gas-tight seal around an instrument inserted into the lumen of said port; and
a lower seal capable of preventing the passage of air or other gaseous through the lumen when there is no instrument inserted into the lumen of said port.

In one alternative embodiment of the flexible port, the upper housing comprises a seal capable of preventing the passage of air or other gaseous through the lumen when there is no instrument inserted into the lumen of said port, and
wherein a seal comprising having a central aperture that is capable of expanding in diameter in order to provide a gas-tight seal around an instrument inserted into the lumen of said port is provided in the lumen of the distal portion of said port.

As mentioned hereinabove, the flexible port also comprises a retention structure, the purpose of which is to prevent the undesired, accidental removal of the port from the body cavity. In one preferred embodiment, this retention structure is a flexible flange. While other retention elements will be described hereinbelow, the flexible flange is particularly preferred, since in addition to its stabilizing and anchoring function, it also contributes to the gas-tight sealing of the port within the surgical incision.

In another aspect, the present invention further provides a trocar suitable for use in conjunction with flexible port disclosed hereinabove, wherein said trocar is characterized by comprising an external circumferential step located close to the distal end of said trocar, and wherein the size and shape of said step is such that it is capable of docking with the internal circumferential step of said flexible port.

In yet another aspect, the present invention is also directed to a kit comprising:
a surgical retractor as disclosed hereinabove;
a flexible port as disclosed hereinabove; and
a trocar as disclosed hereinabove.

The present invention is also directed to a method for retracting tissues, organs or other structures in a laparoscopic or minimally-invasive surgical procedure comprising the steps of:
a) introducing a laparoscopic retractor into the body cavity containing said tissues, organs or other structures,
wherein said retractor comprises an elongate shaft having two or more arms at its distal end, a mechanism for controlling the mutual separation of said arms located at its proximal end, and a membrane attached to said two or more arms, and
wherein the distal arms are in mutual contact when said retractor is introduced into said body cavity;
b) operating said mechanism for controlling the mutual separation of said distal arms, such that said arms separate from each other by a desired amount, thereby causing said membrane to form a non-planar surface that is suitable in shape for retracting said tissues, organs or other structures;
c) manipulating the proximal end of said elongate shaft in order to cause said non-planar membrane surface to move or retain said tissues, organs or other structures; and
d) optionally anchoring said retractor to one or more structure outside of the patient's body by means of one or more straps connected to the elongate shaft, each of which are fitted with a clip for attaching to said structure(s).

In one preferred embodiment of the above-disclosed method, step (a) comprises inserting the retractor through a laparoscopic port that has been previously inserted through the body wall into the body cavity being treated.

In another preferred embodiment of the above-disclosed method, the surgical retractor further comprises a dual layer sheath enclosing the elongate shaft, and step (a) comprises inserting said retractor directly through a surgical incision, following which said sheath is inflated by means of introducing and expansion fluid thereinto.

### BRIEF DESCRIPTION OF THE DRAWINGS

The present invention is illustrated by way of example in the accompanying drawings, in which similar references consistently indicate similar elements and in which:

FIG. 1A schematically illustrates one exemplary embodiment of the invention.

FIG. 1B schematically illustrates one exemplary embodiment of the invention in its closed state.

FIG. 1C schematically illustrates one exemplary embodiment of the invention in its open state.

FIG. 2 schematically illustrates one open/close mechanism of an embodiment of the invention.

FIG. 3A schematically illustrates one preferred embodiment of the invention.

FIG. 3B schematically illustrates the shape of the distal arms of one preferred embodiment of the present invention.

FIG. 3C provides a top view of the retractor unit of the device of the invention in its expanded state.

FIG. 3D schematically illustrates an alternative embodiment of the retractor unit of the device.

FIG. 3E depicts another alternative embodiment of the retractor unit in its closed position.

FIG. 3F depicts the open conformation of the alternative embodiment of the retractor unit that is shown in FIG. 3E.

FIG. 4A shows one embodiment of an anchoring unit of the device of the invention.

FIG. 4B schematically illustrates the device of the invention inserted into the anchoring unit shown in FIG. 4A.

FIG. 5 schematically illustrates one embodiment of the control unit of the device of the invention with the rotating cylinder elements.

FIG. 6A schematically illustrates the control unit of the device of the invention as a scissor like instrument.

FIG. 6B provides a detailed view of the embodiment of FIG. 6A.

FIGS. 7A-7D illustrate an exemplary binary locking mechanism for maintaining the arms of the device in their open and closed positions, and for allowing the movement of said arms from one position to another.

FIG. 8A schematically illustrates one embodiment of an access sleeve that is suitable for use with the present device.

FIGS. 8B and 8C show a further embodiment of a sleeve comprising two detachable halves.

FIGS. 8D and 8E provide two views of a sleeve integrated into a single structure with an anchoring unit.

FIG. 9 schematically illustrates the device of the invention inserted into the body adjacent to a standard port, showing a special sealing plate covering the device of the invention.

FIG. 10 illustrates the use of a device of the invention in its closed conformation as a hook-ended grasper.

FIG. 11 depicts a preferred anchoring unit of the present invention comprising a fabric strip fitted with a clip at its distal end.

FIG. 12A schematically illustrates an embodiment of the retraction device of the present invention in which the distal tips of the distal arms are mutually connected by means of a hinge joint.

FIG. 12B provides a top view of the embodiment shown in FIG. 12A.

FIG. 13A shows a general view of a laparoscopic port of the present invention suitable for use together with the presently-disclosed retractor devices.

FIG. 13B provides a longitudinal section view of the port shown in FIG. 13A.

FIG. 14 provides another view of the laparoscopic port of the present invention.

FIG. 15 provides a cut-away view of the upper seal that is situated in the proximal portion of the port.

FIG. 16 is a longitudinal section view of the tip region of the port of the present invention.

FIGS. 17A to 17C illustrate three different degrees of opening of a typical embodiment of the present invention that comprises a mesh barrier.

FIGS. 18A to 18F depict various different degrees of opening of an embodiment fitted with a scissor/ratchet control mechanism.

FIGS. 19A to 19C illustrate various angled bends and joints within the shaft of the device of the invention.

FIGS. 20A and 20B illustrate a trocar for use with the port of the present invention.

FIG. 20C illustrates a trocar (as shown in FIGS. 20A and 20B) inserted invention inserted within a port of the present invention.

FIG. 20D illustrates the external step in the distal region of the trocar of the present invention.

### DETAILED DESCRIPTION OF PREFERRED EMBODIMENTS

There is, therefore, provided according to the present invention, a novel type of self-retaining tissue retractor, inserted into a body cavity while in a contracted conformation (i.e. having a much smaller size than while in the open conformation). Following delivery to the desired surgical treatment area, the closed device is opened, thereby allowing the surgeon use the device to retract organs, thereby creating a discrete workspace (the surgical window).

The purpose of the device of the invention is to widen the access area to the treatment site (the surgical field), to retract the surrounding tissues, to maintain the resultant size of the access area and to protect the retract tissue or organs by shielding them with the sheet.

### Additional Purposes of the Invention Are:

### Reduction of the Gas Insufflation Pressure

### Obviation of the Need for the Trendelenburg Position (or at Least Use of a Lower Incline).

The device of the invention allows the surgeon to comfortably and safely work within a wide space surgical window. As a result, procedures may be more effective, shorter and safer. As mentioned, the gas insufflations pressure may be reduced. The Trendelenburg position may be unnecessary or at least used with a lower incline. The length of one incision may be reduced. The procedure cost may be reduced.

In its most general form, the device comprises a pair of arms provided by wires or struts. In one preferred embodiment, said wires or struts are joined together at their proximal ends and freely movable at their distal ends. In another particularly preferred embodiment, the distal ends of each of the wires or struts are mutually joined (e.g. by way of a connecting piece of tubing or another type of joint). A particular advantage of this embodiment is that the smooth rounded leading edge of the device that results from the mutual joining or apposition of the free distal ends of the arms facilitates the advancement of the device through the body wall being treated without becoming accidentally engaged or ‘hooked’ on a particular tissue or structure. A retractor sheet or membrane is attached between the distal portions of the two wires, such that upon mutual separation of said distal portions, the sheet is brought into its open position, thereby creating a three-dimensional barrier, similar in form to the palm of a human hand, which may be used for the retraction of organs and tissues during a surgical procedure.

The present description is directed to a method and apparatus for improving the surgical window during minimally invasive (endoscopic) surgical procedures by means of using a self-retaining tissue retraction device that may be inserted into the surgical treatment area in a contracted state. Following insertion, the device will be opened within the body. The device prevents surrounding tissues and organs from penetrating into the surgical window and interfering with the surgeons' view of the treatment site or access into the site.

The device of the invention may be considered as comprising four conceptual units: a retractor unit, an anchoring unit a control unit and an access unit.

According to one embodiment of the invention, the retractor unit comprises two distal arms (provided by wires or struts) which may be moved between open and closed conformations, and a retractor sheet attached therebetween. The proximal ends of the distal arms are continuous with, connected to, or contained within a central shaft which connects the distally-located retractor unit with the proximally-located control unit.

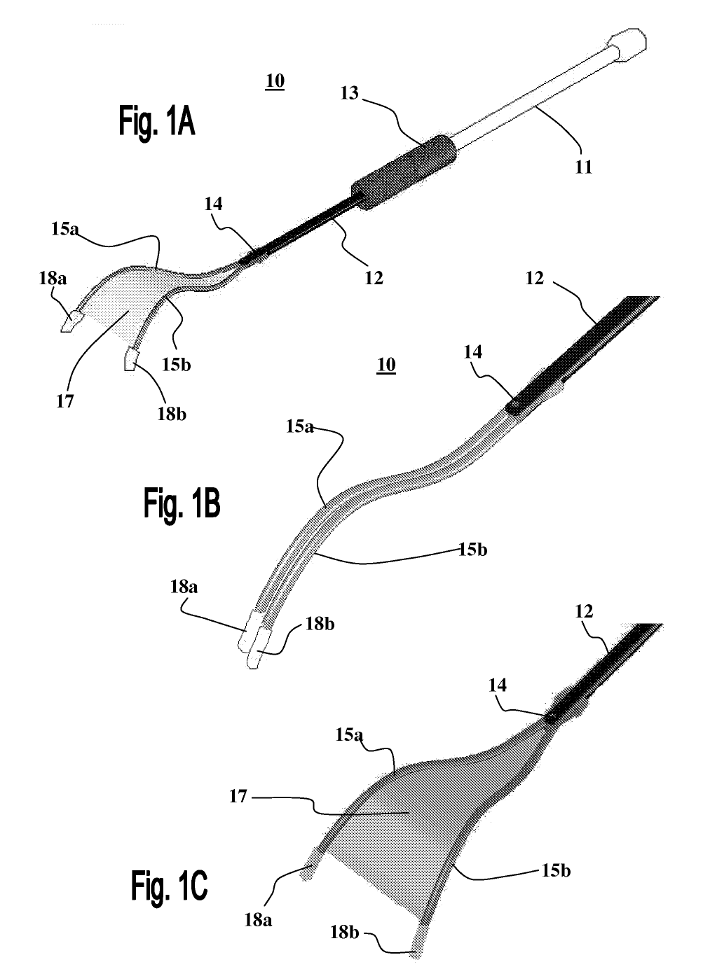

Referring now to the drawings, FIG. 1A illustrates the disposition of the various components of this embodiment of the device, shown generally as 10, wherein a thin barrier or membrane 17 is attached to the curved distal portions of the two arms 15a and 15b. The proximal ends of said arms meet at pivot 14 which is contained within common shaft 12, which has a rectangular cross section and consists of an upper plate and a lower plate. In turn, common shaft 12 is connected at its proximal end to a tubular shaft 11, which extends to the proximal extremity of the device.

It should be noted that the term “proximal” as used herein in relation to the device of the invention refers to the side, direction or extremity closest to the operator. Thus, the proximal extremity of the device always remains outside of the body of the patient being treated. Conversely “distal” refers to the side, direction or extremity that is furthest away from the operator, which is towards the center of the patient's body.

Returning now to FIG. 1A, the device 10 may also include an a control unit 13 surrounding portions of both the common shaft 12 and tubular shaft 11, the purpose of said unit being to cause the arms 15a and 15b to move between their open and closed conformations. One embodiment of the control unit will be described in more detail hereinbelow. Finally, it will be noted that in this embodiment, the distal extremities 18a and 18b of arms 15a and 15b, respectively, are coated with silicone or polyurethane or another similar material, in order to prevent said extremities from causing trauma to the patient's soft tissues.

In the closed conformation of the device 10, the arms 15a and 15b are closely apposed, one to the other, as shown in FIG. 1B.

In the fully-opened conformation of the device, as shown in FIG. 1C, the distal ends of the metal arms 15a and 15b each typically have a length of 100 mm, with an angle of up to 180 degrees therebetween. However, devices with any other suitable length and separation angle may also be used, and hence fall within the scope of the present invention. Said arms are spread far apart from each other, with a covering sheet or membrane 17 lying between them thus creating a triangular form, similar to human hand. It is to be noted that the retractor sheet does not cover the lateral sides of the arms, thereby allowing their use in tissue and organ grasping (in addition to the retraction property of the sheet).

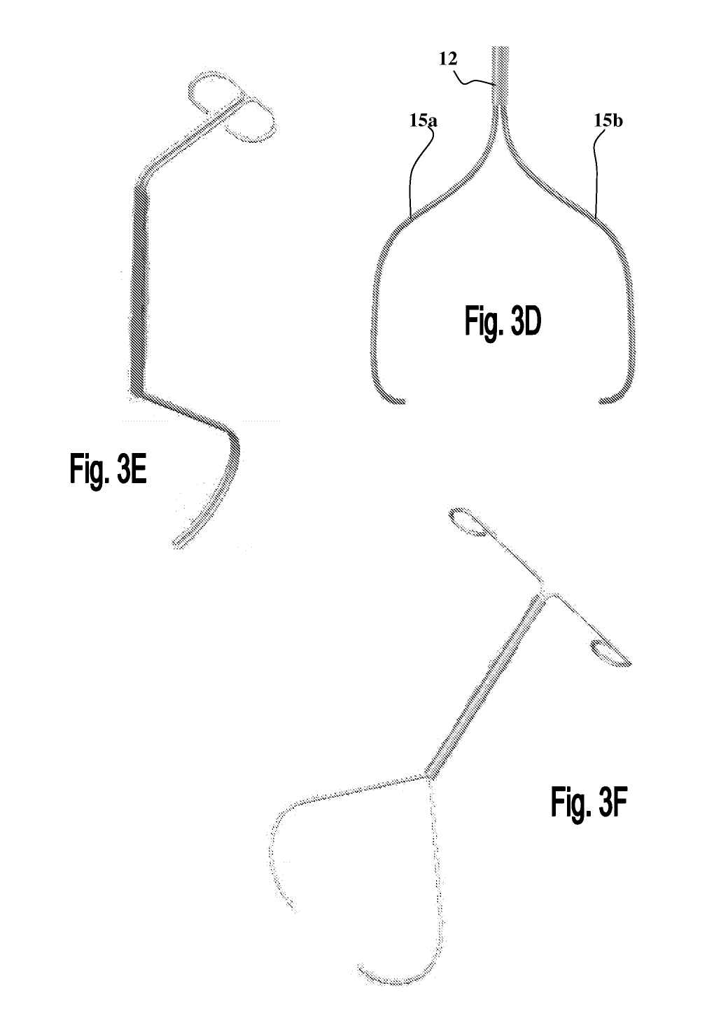

In one preferred embodiment of the invention, the arms are curved, which together with the barrier membrane in its open conformation create a hand-like device having a concave surface on one side, and a convex surface on the other side, as shown in FIGS. 3A, 3B and 3C. In some embodiments, the curvature of the arms may be modified by the surgeon, who may bend said arms into any desired form, thus altering the degree of convexity or concavity. In one embodiment, the arms of the device are each formed into an “L” shape. It is to be noted that the distal arms of the device may be constructed in any desired form, such that said arms (and hence the barrier membrane attached thereto) when fully open can adopt any desired outline shape (e.g. circular, elliptoid, rectangular, square, polygonal etc.). In addition, the depth of the curvature of the hand-like structure formed by the barrier membrane stretched over the distal arms in their open state may also be pre-determined by means of manufacturing the device with distal arms having a particular shape, or alternatively, by means of the operator manually adjusting this shape immediately before or during the operative procedure.

In one preferred embodiment, the device is used with the convex side of the arms facing the organs that are to be retracted. Alternatively, in other preferred embodiments, it may be found useful to employ the device in the opposite direction, i.e. with the concave side facing the organs to be retracted.

In another embodiment of the device, the arms may be curved into the shape depicted in FIG. 3D. The arm shape shown in this figure permits the creating of a wider surgical field than is possible with the shape shown in FIG. 3C.

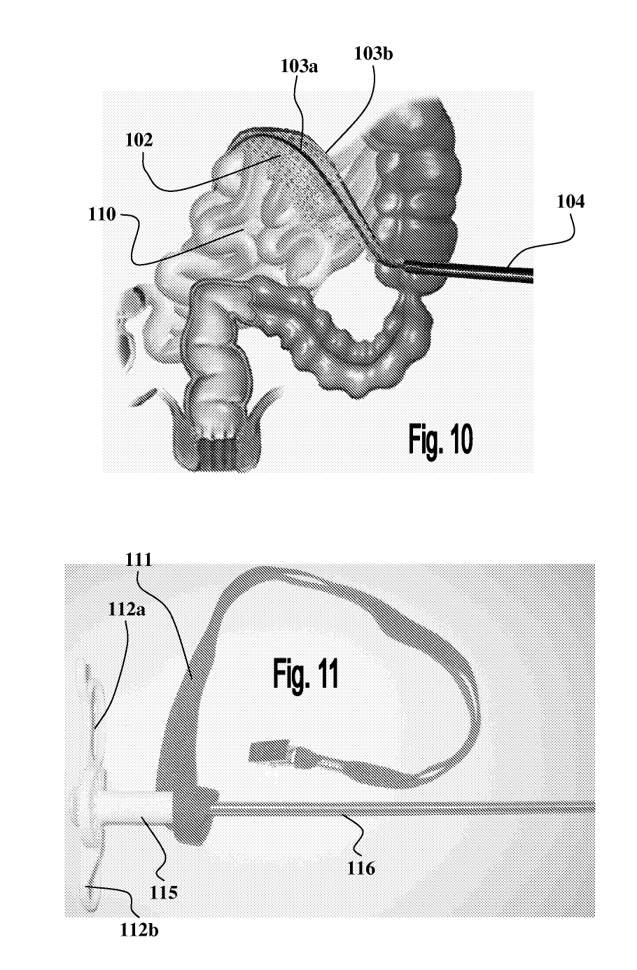

It is to be noted that in addition to its retraction function, the device of the present invention may also act as a grasper by means of closing the arms (in their closed conformation) around the tissue or organ to be held, such that said tissue or organ becomes trapped within the barrier membrane or mesh. This particular use is illustrated in FIG. 10, in which a preferred embodiment of the device of the invention comprising shaft 104 and distal arms 103a and 103b (in a substantially closed state) is being used to grasp small intestinal loops 110. It will be noted that in this conformation, although the barrier sheet 102 (here shown in the form of a mesh) is in a flaccid state, it provides a degree of cushioning that serves to prevent the small intestine from being traumatized by the distal arms.

Furthermore, in another preferred embodiment of the invention, the retraction device in its closed conformation may be used as a hook probe in order to move or drag structures such as intestinal loops.

A still further use of the device of the invention (particularly applicable when the barrier membrane is constructed of a mesh material) is a form of collection basket. In this embodiment, the retraction device in its open conformation is brought into close proximity with an isolated or detached structure or object (e.g. excised portion of a tissue or organ, or a non-physiological object such as a swab, staple etc.). The device is then fully closed, thereby enclosing said structure or object within a basket-like space completely enclosed by the barrier membrane. The structure or object may then be moved from place to place within the body cavity and/or completely removed from the body.

In another preferred embodiment of the invention, each of the arms of the device has an angled profile as shown in FIG. 3E. It may be seen in this figure that each half of this embodiment also has a scissor like upper portion that is continuous with a middle straight rod section, which in turn is continuous with the distally placed arms. Thus, in this particular embodiment, each half of the device may be formed from a single rod or wire extending from the proximal scissor-like handle to the distal arm. The straight rod sections of each of the two halves are bound together by silicon tubing 12 (or a similar tubular element), as shown in FIG. 3D, thereby allowing each rod to rotate in relation to its partner. In addition to this hinge-like function, the silicon tubing also contributes to the gas-tight sealing of the device within the surgical incision, thereby helping to prevent gas leak from the body cavity.

In another embodiment of the retraction device of the invention, the shaft of said device is enclosed by a dual-layer sleeve, wherein said sleeve may be caused to inflate in a balloon-like manner by means of introducing an expansion fluid (e.g. air or water) into the space between the two layers of said sleeve. In one preferred implementation of this embodiment the dual-layer sleeve is made of silicone. Inflation of the sleeve (in the manner described above) is facilitated by the presence of an inflation tube extending proximally, into which the expansion fluid may be introduced using a syringe or pump device. The expandable silicon sleeve is particularly advantageous when the device of the invention is to be passed through the body wall without the use of a laparoscopic port. In such an embodiment the retraction device fitted with the aforementioned sleeve is directly inserted through an appropriately sized surgical incision. The sleeve is then expanded, thereby simultaneously causing in situ stabilization of the device and providing gas-tight sealing of the incision around the device.

The angled arm profile permits the device to be opened wide, thus causing the retraction sheet or membrane attached between the two arms (omitted for clarity in FIGS. 3E and 3F) to become tightly stretched. The curve at the end of each arm is used to create a three-dimensional, hand- or scoop-shaped device shape that can retract organs and tissues that have an uneven, fluctuating surface topography. This is used to create a tangent motion of the device relative to the curvature of the surface.

In a particularly preferred embodiment of the device of the invention, the distal arms are shaped such that the distal tips of each of the distal arms are always in contact (or very nearly in contact) with each other—regardless of whether the device is in its fully open or fully closed conformation or in some conformation between those two extremes. In this way, the two distal arms when in an open conformation describe a continuous loop (albeit a complex, three dimensional loop) and therefore do not possess free distal extremities, thereby eliminating the possibility of causing tissue damage by said extremities.

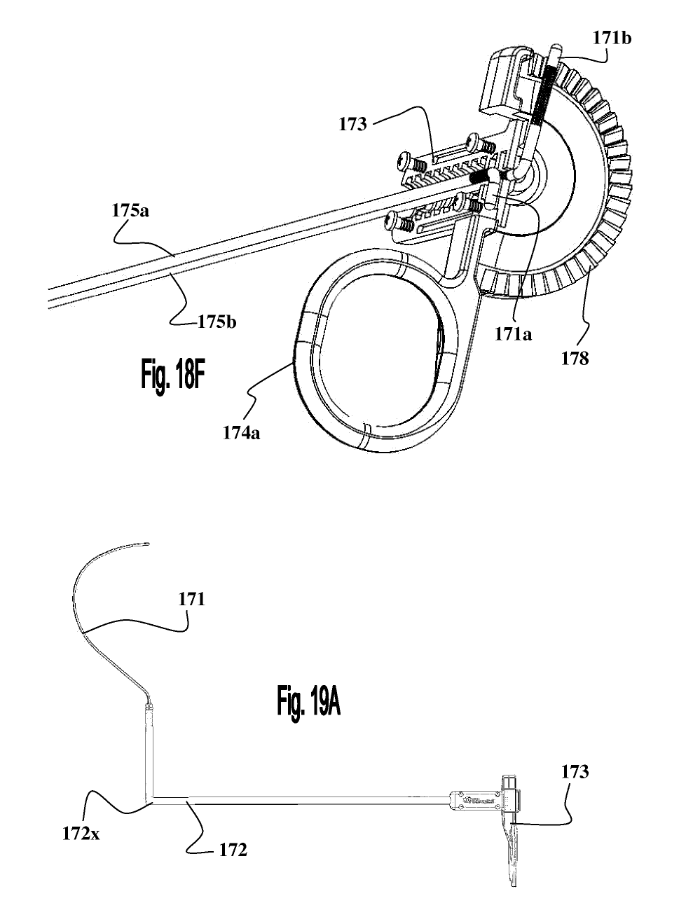

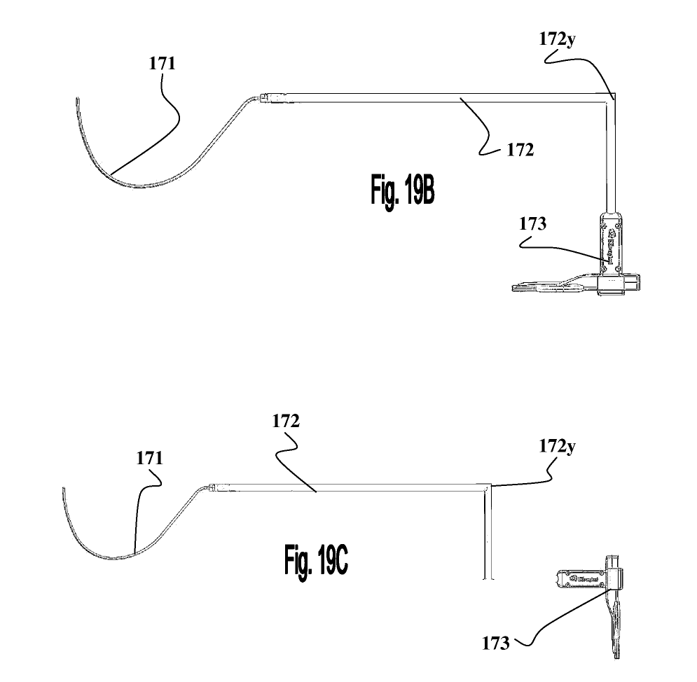

In one version of this embodiment, the distal tips of the distal arms are not actually connected to each other, but remain either in actual contact or nearly in contact with each other. An example of this particular embodiment may be seen in FIGS. 18B to 18D, where the distal tips of the two distal arms are represented as being extremely close to each other, regardless of the degree of opening of the device.

In another version of this preferred embodiment, the distal tips of the two distal arms are connected by means of a flexible hollow connector, preferably a length of silicon tubing. In a still further version, said distal tips are mutually connected by means of a dedicated joint. One example of such a joint is shown in FIG. 12A, in which the two distal arms 122a and 122b are movably joined together at their distal tips by means of joint 121. A plan view of this arrangement is shown in FIG. 12B.

The essentially rigid portions of the device (e.g. the wire arms and shafts) may be constructed from metals such as stainless steel 316 or 304 or ph 17-4, or any suitable biocompatible polymer including (but not limited to) polycarbonate and Ultem.

In one embodiment of the device of the invention the barrier sheet lying between the arms is made of a non-compliant material such as (but not limited to) silicone.

In another embodiment of the device, the barrier sheet may be made of a compliant material, or from a combination of non-compliant and compliant materials. Examples of suitable compliant materials include (but are not limited to) biocompatible materials such as polyamide, polyethylene, polyurethane and an adhesive material for attaching said sheet to the arms. The barrier sheet may be constructed from these materials as a continuous sheet, as a pad or in the form of a mesh.

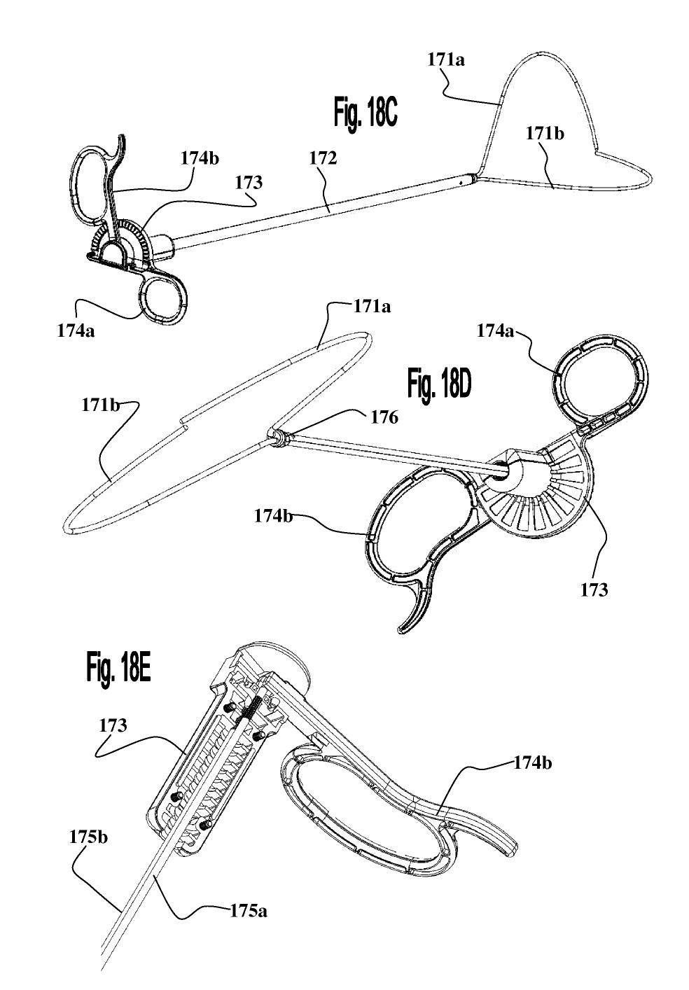

In a particularly preferred embodiment, the barrier sheet consists of a polyurethane mesh. An example of this embodiment is illustrated in FIGS. 17A to 17C, which show a retractor device of the present invention having the distal tips of the distal arms positioned in contact with each other (as explained hereinabove), and wherein a polyethylene mesh barrier sheet 177 is connected to said arms. In FIG. 17A, the device is shown in its fully closed conformation, while FIGS. 17B and 17C show progressively greater degrees of opening. It will be appreciated that the use of distal arms such as those shown in these figures together with scissor-like ratchet control mechanism (as described hereinbelow and illustrated in FIGS. 18A to 18E) provides the operator with the ability to create in situ a hand-like laparoscopic retractor having any desired degree of opening. In this way, the shape of the barrier membrane surface may be adapted to the particular tissues and organs that require retraction or movement.

The above-described mesh barrier may also, in some embodiments, be manufactured from other materials, including (but not limited to) polyester, polyurethane, cotton, nylon 6, nylon 6/6, silicone or polypropylene.

In one preferred embodiment of the device, the barrier membrane is transparent, thereby optimizing the operator's ability to see the tissues and structures that are located on the far side of said barrier. Optimal viewing of said tissues and structures may also be obtained by using a barrier sheet that is provided in the form of a large-pore mesh.

The use of the aforesaid transparent membranes and large-pore mesh barriers provides the operator with the capability of viewing the retracted organs and tissues at all stages of the procedure, and thereby enables prompt identification of bleeding vessels and/or trauma to said organs and tissues.

The arms of the may be constructed from any suitable biocompatible material, including biocompatible metals such as stainless steel (e.g. SS 316, 304 or ph 17-4) or of a suitable biocompatible polymeric material such as (but not limited to) Ultem or polycarbonate.

When acting as a retraction instrument, the purpose of the device of the present invention is to hold back the forces and pressures that are applied on the device by the retracted tissues or organs, thereby preventing them from penetrating into the surgical site. The device of the invention is capable of resisting the forces exerted by tissues and organs having a mass of up to 5 KG.

In one embodiment, the barrier sheet is attached to the arms by means of inserting said arms into folded ends formed in the sides of said sheet. Following assembly in this way the sheet folded ends are glued to the arms using an appropriate biocompatible adhesive, such as a silicon glue or polyurethane film.

In an alternative embodiment, the barrier sheet is assembled into the aforementioned folded ends, as described above, but is not glued thereto.

In yet a further embodiment, the barrier sheet may be attached to the distal arms of the device by means of welding. In a still further embodiment of this aspect of the invention, the barrier sheet may be attached to the distal arms by means of inserting said arms into a tube (e.g. constructed of polyurethane, silicone, PVC, PTFE, FEP, PFA etc.) that has been attached to the perimeter of said sheet by means of sewing, welding or gluing.

In another embodiment of the invention, the barrier sheet component may be made of a plastic mesh or thin metal instead of plastic sheet or a combined metal mesh with transparent covering plastic sheet.

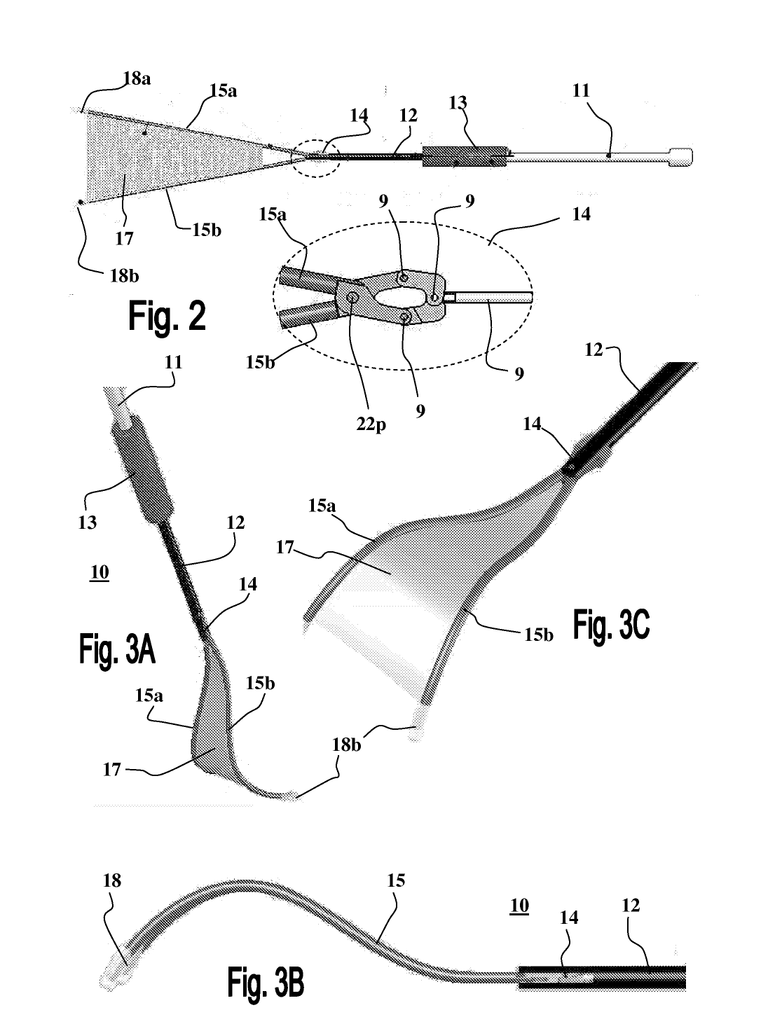

In one alternative embodiment, the arms are connected to the central rod by means of a pivot joint and can be opened and closed in scissor motion. An example of such an embodiment is shown in FIG. 2, in which the pivot joint 14 is shown in more detail in the enlarged view provided in that figure. It may be seen from this enlarged view that pivot joint 14 is actually formed from a primary pivot 22p together with a series of supplementary pivots 9. In this embodiment of the invention, the retractor unit in its closed state including the arm and the joint preferably has a diameter of 5 mm or less.

In another embodiment the retractor's arms and sheet can be formed into sizes and shapes different from those exemplified in this disclosure without deviating from the scope thereof.

In one preferred embodiment, the shaft of the device has a diameter in the range of 0.5 to 15 mm, preferably 6 mm and a length in the range of 3 cm to 1 m, preferably 30 to 40 cm.

In some embodiments, the shaft of the device is hollow and contains within its internal cavity an internal rod that is used to control the opening and closing of the arms in a gas-tight manner, such that that insufflation gas will not be released from the entry port. In addition, the shaft may be coated with silicon, thereby permitting complete sealing of the incision.

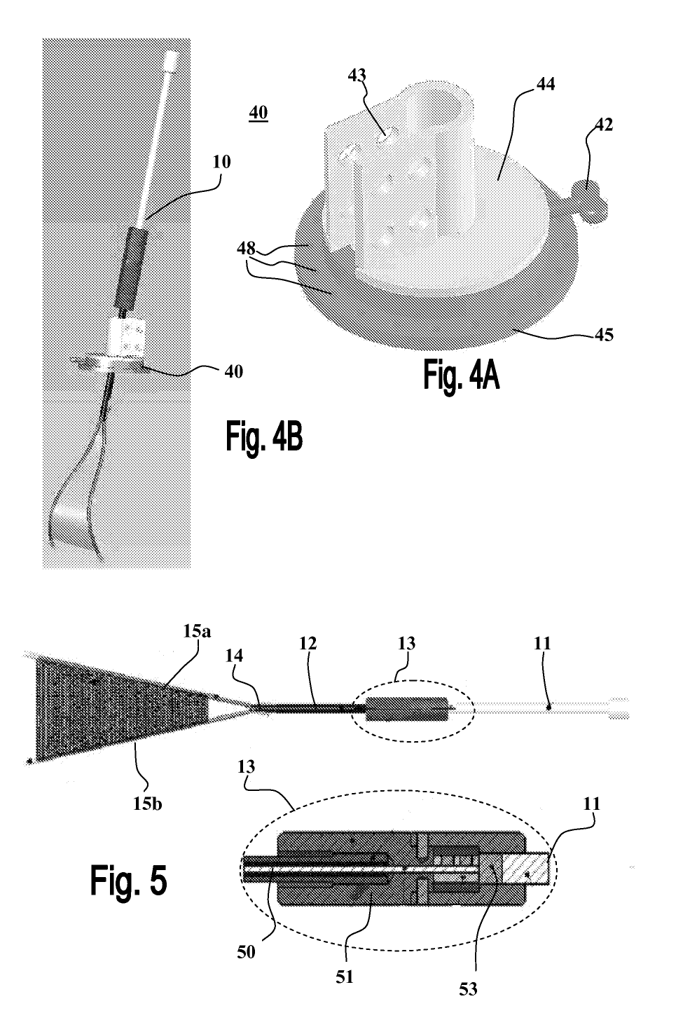

In some cases, the device of the invention may be kept in position in situ by means of an anchoring unit, one embodiment of which (shown generally as 40) is illustrated in FIG. 4A.

The first part of the exemplified anchoring unit is the generally circular attachment plate 45, which in use may be attached to the outer surface of the tissues surrounding the surgical incision by means of stitches through the sewing holes 48 around the plate. In alternative embodiments of this aspect of the invention the attachment plate may be attached by means of glue or vacuum.

A second key part is the rotating round plate 44 located within the attachment plate 45 extending out from it and clamped thereto with a locking screw 42, thereby permitting rotation of the plate in relation to the attachment plate. On top of the rotating plate stand two vertical square holding plates 43, which can be tightened by screws in four holes formed in each of said plates, in order to clamp and immobilize the shaft of the device.

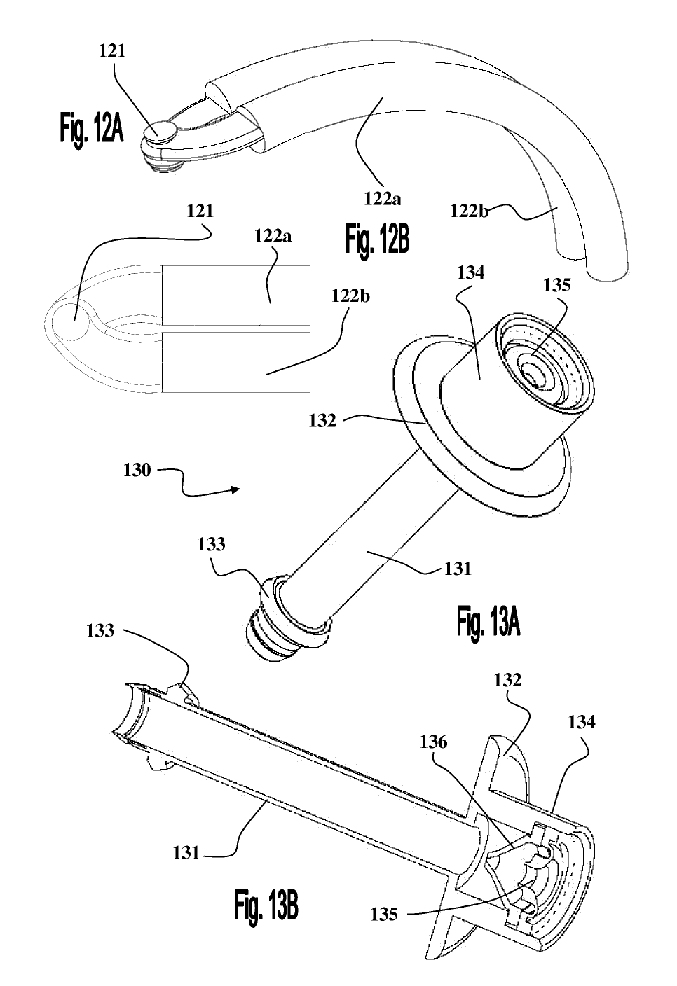

FIG. 11 illustrates an alternative highly preferred embodiment of the anchoring unit, in which said unit is provided in the form of a soft strip 111 tied (or otherwise attached) to the central shaft 116 of the retraction device. In the example shown in the figure, the strip is attached to the shaft at its upper, proximal end, close to (and/or in physical contact with) a scissor-like control unit 115 (described in more detail hereinbelow) comprising two scissor-handles 112a and 112b. The distal end of the strap is attached to a clip which may be used to anchor said strap (and hence the retraction device) to surgical drapes, sheets, operating table or any other sterile object located outside of the patient's body. The strap used in this anchoring unit may be constructed of silicone or any other suitable biocompatible polymer of fabric, while the clip may be fashioned from stainless steel or a biocompatible polymer such as polycarbonate. In addition to the use of a single strap as illustrated in FIG. 11, in many cases, it will be found advantageous to use a plurality of such straps (preferably—but not limitatively—between two and six straps). The proximal ends of all of said straps may be tied or otherwise attached to the shaft of the retraction device at the same place, while the clips located at the distal ends thereof permit anchoring of said device to widely-spaced objects close to the patient, thereby increasing the anchorage stability. The aforementioned straps may be custom-made for use with the presently disclosed retraction device. Alternatively, commercially available generic clip/strap assemblies may be used.

It is to be recognized that the attachment units described in the foregoing passages are only illustrative examples of such units: various other mechanical solutions may also be used without deviating from the scope of the present invention.

One embodiment of the control unit (mentioned in passing hereinabove) shown in FIG. 5 combines a static cylinder element 51 and a dynamic cylinder element 53. Rotation of the dynamic cylinder control (by means of rotation of the tubular shaft 11 that is connected thereto) causes an internal rod 50 to move up or down within the shaft, thereby causing the opening and closing of the arms. The details of this exemplary mechanism are shown in the enlarged view 13 provided in FIG. 5.

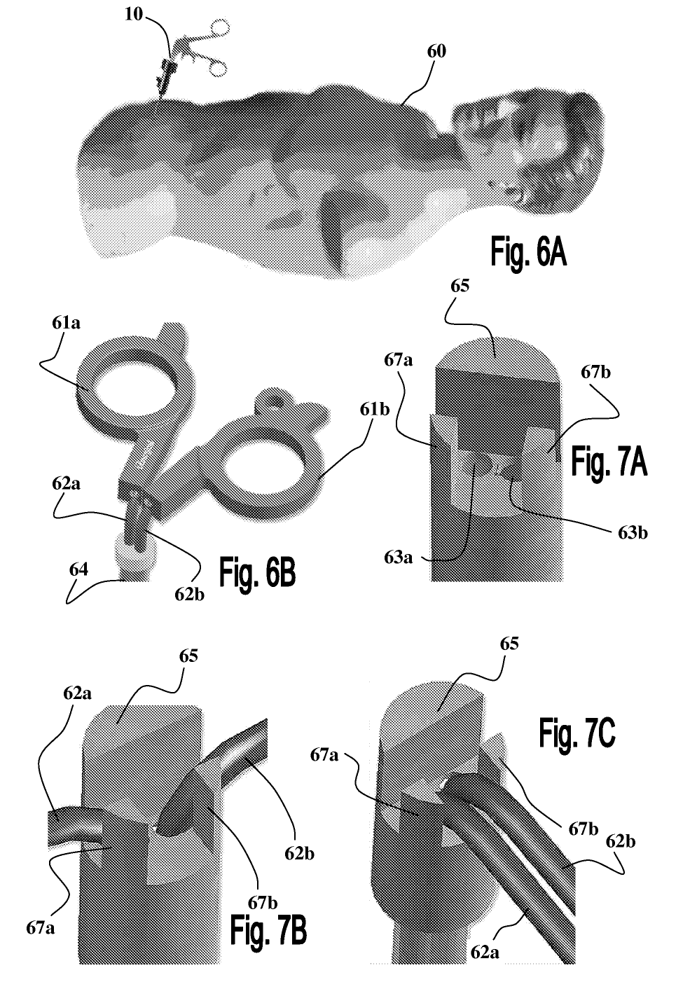

In a further highly preferred embodiment, the opening/closing mechanism of the control unit will be constructed as a scissor like unit, as shown generally in FIG. 6A. A more detailed view of the scissor-like unit is provided in FIG. 6B, in which the proximal ends of the wires 62a and 62b are shown ending in scissor handles 61a and 61b, respectively. Said wires run proximally through hollow shaft 64, ending in the distal arms (not shown in this figure). A ratchet mechanism may be used to lock the scissors handles (and hence the distal arms) in any desired position.

An example of a particularly preferred embodiment of a scissor-like unit employing a ratchet mechanism is shown in FIGS. 18A to 18F. The particular ratchet mechanism used in this embodiment is constructed such that the distal arms of the retractor device may adopt any of the following positions:
fully closed state (i.e. having an angle between the distal arms close to 0 degrees);
fully open state (i.e. having an angle between the distal arms of 180 degrees);
26 intermediate open positions.

This particular type of mechanism is highly advantageous since it allows the operator to control the degree of opening of the distal arms—and hence the three-dimensional shape of the barrier membrane—to a high degree of precision. The mechanism itself comprises a fixed scissors handle 174a to which one of the wires is connected, and a movable handle 174b to which the other wire is connected. As explained above (with reference to FIG. 6B) these wires run distally from the scissor-handle unit through a guiding tube (shown in FIGS. 18A to 18D as 172), ending in distal arm wires 171a and 171b, respectively. The movable handle 174b may be rotated about its pivots such that it engages with the teeth of ratchet mechanism 173. It will be seen that in FIG. 18A the closed position of the scissor handles 174a and 174b result in the corresponding closure of distal arms 171a and 171b. In FIG. 18b, however, the movable handle 174b is moved about one third of the way toward the fully open position, thereby causing a corresponding opening of distal arms 171a and 171b. Increasing degrees of opening are illustrated in FIGS. 18C and 18D. Further details of the ratchet mechanism are illustrated in FIGS. 18E and 18F, which show cut-away views of said mechanism. For the sake of clarity, guiding tube 172 is omitted from these two latter drawings, thereby revealing the positions of control wires 175a and 175b. In addition, FIG. 18F provides an enlarged view of the connections of the proximal ends of said wires 175a and 175b to the scissor handles 174a and 174b, respectively.

In order to operate the scissor-like mechanism described immediately hereinabove, it is necessary to first lift the movable handle 174b in order to separate it from the ratchet teeth located on the fixed part of said scissors mechanism. The movable handle is then moved to the desired position and then released from lifted position such that the ratchet mechanism once more locks the handles into position, thereby preventing further rotation.

The scissors handles and ratchet mechanism may be constructed from any suitable material, but are preferably constructed from a medical grade plastic such as polycarbonate, Ultem, nylon, polypropylene and so on.

In some preferred embodiments of the device of the present invention, said device incorporates one or more angled bends or joints in the control wires and/or shaft, in order to obtain retractor units disposed in a particular direction or angle. One example of such an embodiment is illustrated in FIG. 19A in which shaft 172 is bent at right angles at about one third of its length from its distal end 172x. A scissor-like control unit 173 is also shown attached to the proximal end of shaft 172. Assuming that the long portion of shaft 172 is inserted vertically (i.e. at right angles to the body wall of the patient), the right angled shaft bend will result in the distal arms 171 of the retractor unit and the attached barrier membrane (not shown) adopting a conformation that is generally parallel to the body wall.

While the device shown in FIG. 19B also incorporates a right-angled shaft bend, the position of this bend 172y is approximately one third of the distance from the proximal end of shaft 172. This arrangement permits the placement of distal arms 171 (and their attached barrier membrane; not shown) at a much greater distance from the insertion port than in the arrangement shown in FIG. 19A. Finally, FIG. 19C illustrates a further embodiment, wherein the scissor-handle unit 173 is removable from the proximal end of shaft 172. This is particularly useful in procedures wherein a large number of ports and/or laparoscopic instruments are being used simultaneously, and wherein, therefore, it is desirable to remove as much of the proximal handle as possible, in order to maximize visualization and reduce interference between different instruments.

The angled bends described immediately hereinabove may be achieved at any point along the shaft of the device, and more than one such bend may be incorporated within a single device. The angled bend may be pre-formed and fixed, or alternatively may be provided by an adjustable hinge joint or universal joint between two sections of the shaft.

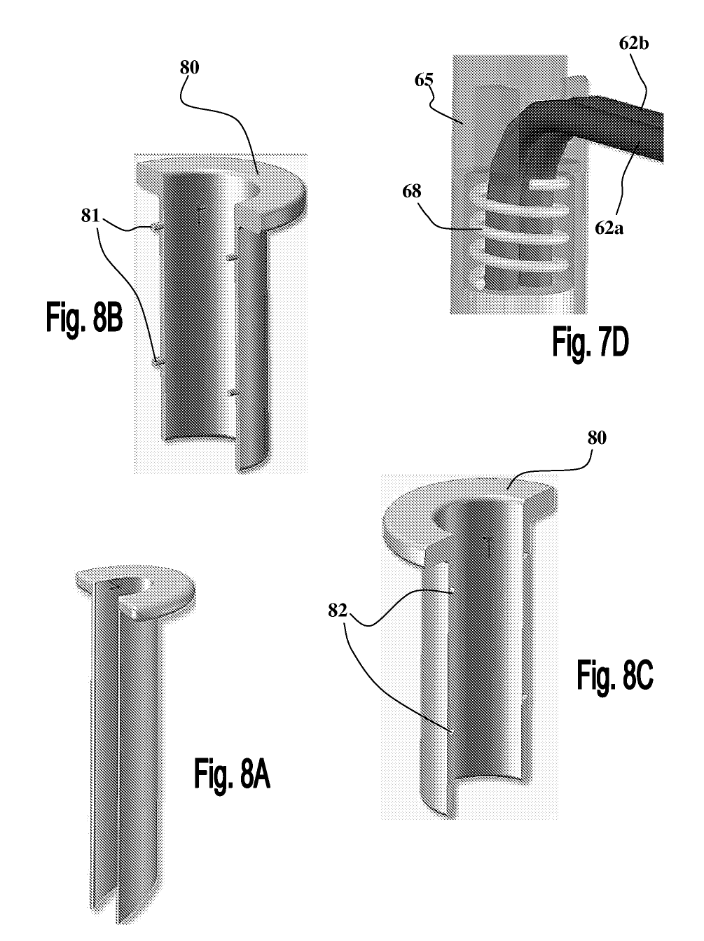

In an alternative embodiment, the device may comprise a binary locking mechanism for locking the arms of the device in either the open or the closed conformation. This mechanism has two states:
1. Closed state (the arms are mutually apposed) as shown in FIG. 7C.
2. Open state (the device is unfolded) as shown in FIG. 7B.

The locking mechanism (shown in more detail in FIG. 7A with the arms removed for clarity) comprises a generally cylindrical element which is divided at its upper end into a single posterior portion 65 and two anterior portions 67a and 67b. Said posterior and anterior portions are separated by a groove in which the proximal wires or arms of the device 62a and 62b are housed when in the aforementioned open state, as shown in FIG. 7B. In addition, the locking mechanism also possesses a second groove located between its two anterior portions 67a and 67b, the purpose of said groove being to house the proximal portions of arms 62a and 62b, when said locking mechanism is moved into its closed state. As shown in FIGS. 7A to 7C, the vertical portions of arms 62a and 62b are contained within separate channels, 63a and 63b, respectively. The lower ends of these channels, in turn, open into a larger single channel. As shown in FIG. 7D, this larger channel contains a biasing spring through the central lumen of which run the two arms contains within its lumen surrounding, the purpose of which is to maintain the mechanism in the selected (open or closed) position.

In some embodiments, the present invention may further provide a dedicated access unit, the purpose of which is to facilitate the insertion and passage of the retractor unit through the body wall and into the body cavity being treated.

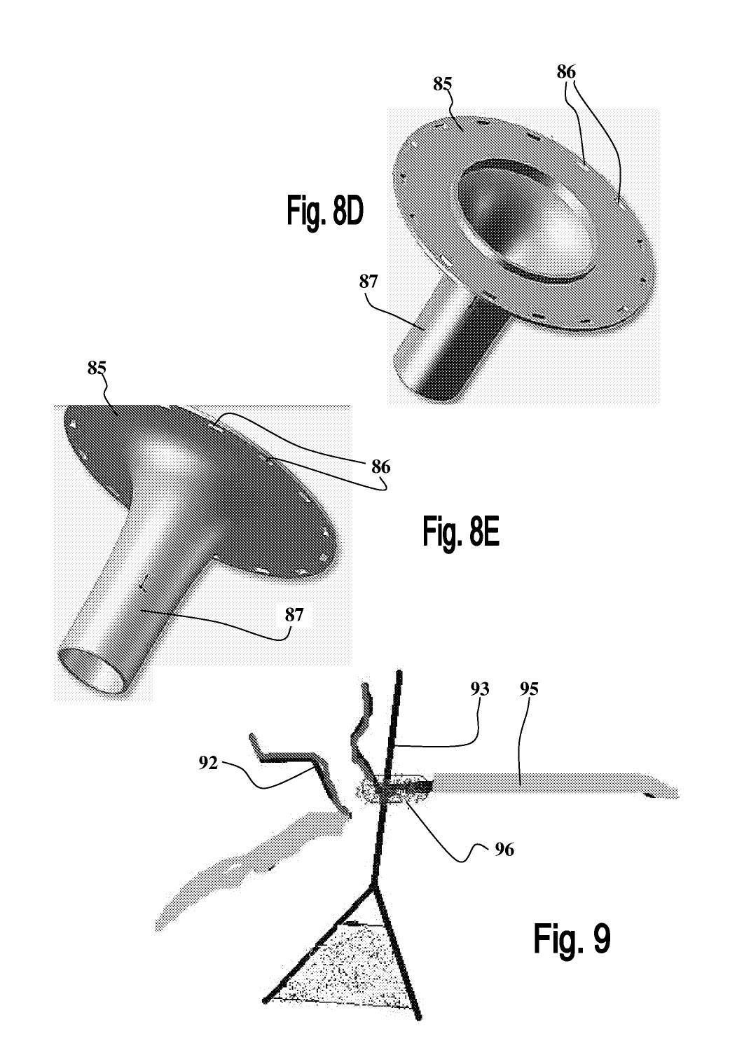

In one preferred embodiment, the access unit of the present invention may comprise a simple sleeve, the purpose of which is to facilitate the insertion and removal of the device from the body. A longitudinal section view of such a sleeve is shown in FIG. 8A. As shown in that figure, the sleeve may have sharp inferior edges in order to assist in penetration through the abdominal wall incision into the abdominal cavity. Thus, after the creating of the initial body wall incision by the surgeon, the sleeve is inserted in order assist in the penetration through the abdominal fascia. The device of the present invention is then inserted through the sleeve and the sleeve can be pulled out. The procedure is then reversed in order to remove the device from the abdominal cavity.

In an alternative embodiment, the sleeve is constructed in two parts that can be assembled in situ by mating two half sleeves. This may be achieved by docking a series of small pegs or protrusions 81 in one half of the sleeve (FIG. 8B) into corresponding holes 82 in the other half (FIG. 8C) In order to remove the sleeve it is disassembled into its two component halves.

In yet another embodiment of the device of the invention, the sleeve structure is incorporated into a single structure together with an anchoring unit, as shown in FIGS. 8D and 8E. It may be seen from these figures that the generally tubular sleeve 87 broadens at its proximal end such that it becomes continuous with anchoring plate 85, in the perimeter of which are formed suture slots or apertures, for the purpose of attaching said plate to the skin of the patient.

In a particularly preferred embodiment, the access unit is provided in the form of a flexible entry port that is specifically designed and constructed for use together with the retractor unit of the present invention. One example of such a port is shown in FIG. 13A. The flexible port 130 illustrated in this figure comprises a hollow shaft 131 the inner lumen of which is continuous with an upper housing 134 on its proximal end and a lower tip at its distal extremity. An upper flange 132, which assists in the stabilization of the proximal part of the device against the skin surface of the patient being treated, is located at the lower (distal) end of said upper housing 134. In the embodiment shown in FIG. 13A, an upper seal 135 is visible within the lumen of housing 134. Turning now to the mid-line longitudinal section presented in FIG. 13B, it may be readily seen that housing 134 actually contains two sealing elements: the aforementioned upper seal 135 and a lower seal 136. The function of upper seal 135 is to prevent leakage of air or insufflation gas while laparoscopic instruments (such as the retraction device of the present invention) are present in the port. The structure of a typical example of this disk valve is shown in more detail in the cut-away view shown in FIG. 15, in which the central aperture 152 and annular ridge 150 be seen. Upon insertion of a laparoscopic tool into the port, aperture 152 becomes enlarged such that said tool fits snugly within said aperture in a gas-tight manner. This expansion in aperture size is facilitated by the compression of annular ridge 150, by the pressure exerted by the inserted tool. Upper seal 135 is typically constructed of a compressible material such as silicone rubber. The purpose of lower seal 136 (illustrated in FIG. 13B) is to prevent the leakage of air or insufflation gas while there no laparoscopic instruments inserted within the port. Many different types of commercially available valves may be used as lower seal 136 including dome valves and cross-slit valves, such as those provided by Minivalve International (Ohio, USA). The key feature of said lower seal is that in its resting state (i.e. when no instrument is present within the port), the seal is completely closed. Both the upper and lower seals are affixed within the housing of the port using a biocompatible adhesive such as silicone glue. In an alternative embodiment of the port of the present invention, the disk valve (upper seal 135 in FIG. 13A) may be present in the distal portion of the tubular shaft 131 (i.e. within the portion that is inserted within the body cavity, close to the distal extremity of the port).

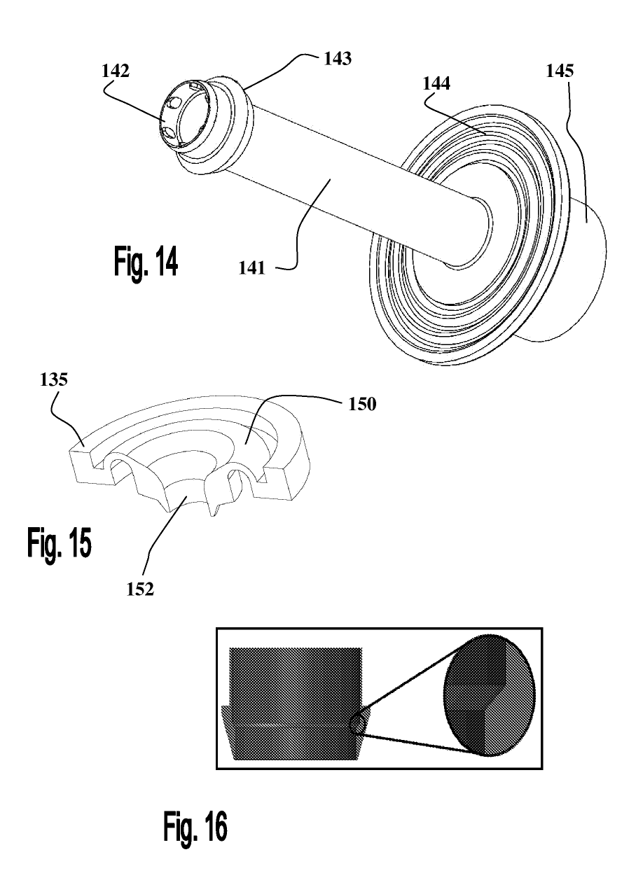

A further structural feature of the port of the present invention is the flexible flange 133 that may be seen surrounding the outer surface of the lower (distal) portion of the hollow shaft 131 in FIGS. 13A and 13B. The flexible flange is also shown (143) in the perspective view presented in FIG. 14. This flange (which may be constructed from silicon or a similar biocompatible polymer) operates as an anchoring element that prevents the accidental removal of the port from the body cavity. In addition to this anchoring function, said flange also assists in sealing the body wall incision (i.e. from the interior surface of the body wall) through which the port was inserted. During insertion of the port into the body cavity the flange is in a collapsed state. Upon entry into the body cavity the flange springs open thereby presenting a large surface area than the area of the surgical incision through which the port was introduced. The port can be removed (e.g. at the end of the surgical procedure) by applying a removal force that is high enough to once more cause deformation or collapse of the flange.

The distance between upper flange 132 and lower flange 133 is generally in the range of about 1 to 30 cm, preferably about 4 to 8 cm. The precise upper flange—lower flange length chosen is determined by the thickness of the body wall of the patient being treated. In certain embodiments, the upper housing 134 and/or the upper flange 132 is constructed such it/they are movable along the hollow shaft 131, in order that the distance between said upper flange 132 and lower flange 133 may be adjusted in situ. The movement of the housing and/or upper flange along the shaft may be a simple sliding movement, or alternatively, the housing and/or upper flange and the shaft may interact by way of a mutually interacting thread, optionally provided with a ratchet mechanism in order to stabilize the device at any desired inter-flange length.

In an alternative embodiment (not illustrated), the distally-located flexible flange anchoring mechanism is replaced by, or supplemented with a folded bellows-like structure which, upon insertion of the port through the body wall will be stretched by the insertion trocar, thereby causing said structure to offer minimal resistance to port entry. However, upon removal of the trocar, the bellows-like structure will revert to its folded conformation, thereby achieving a diameter greater than the insertion incision and thus preventing accidental withdrawal of the port.

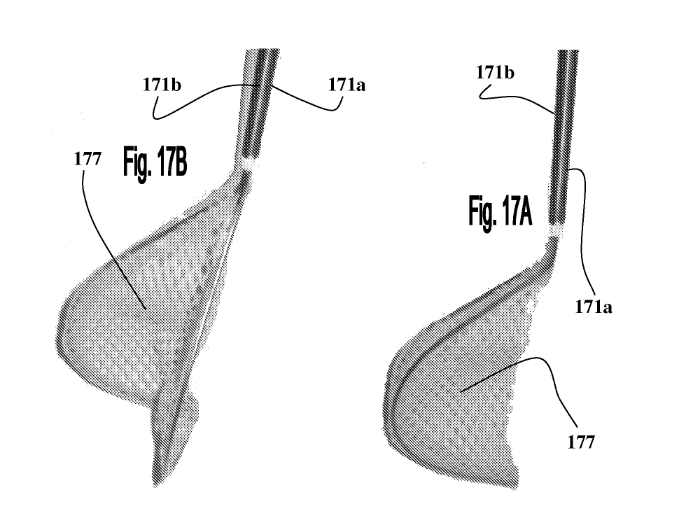

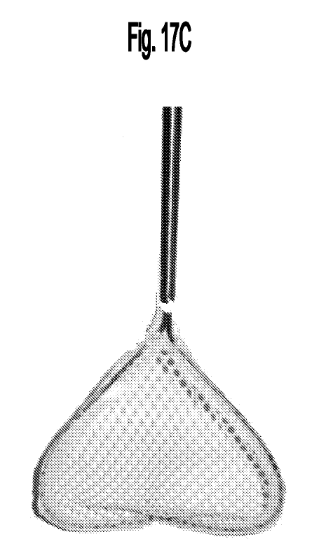

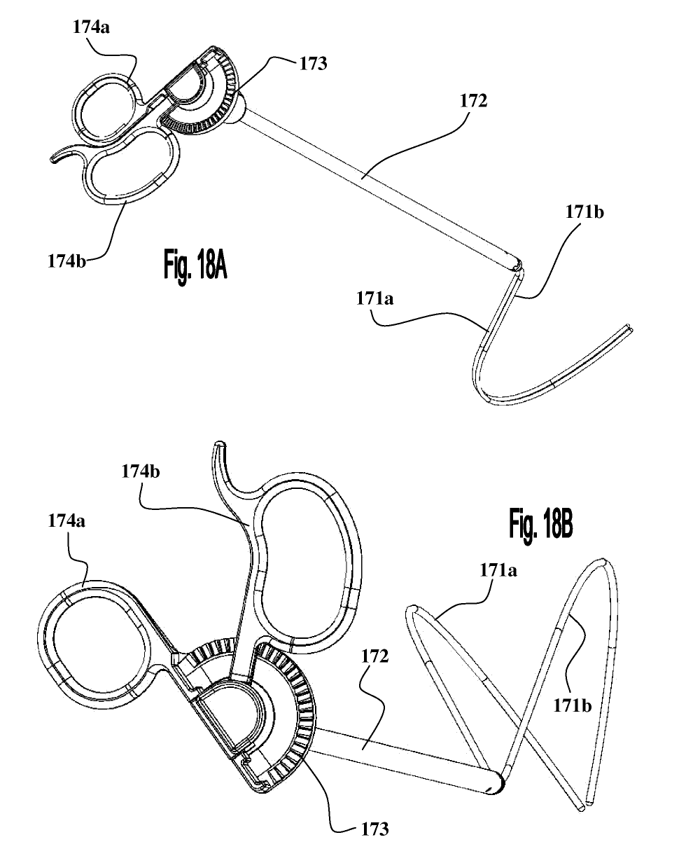

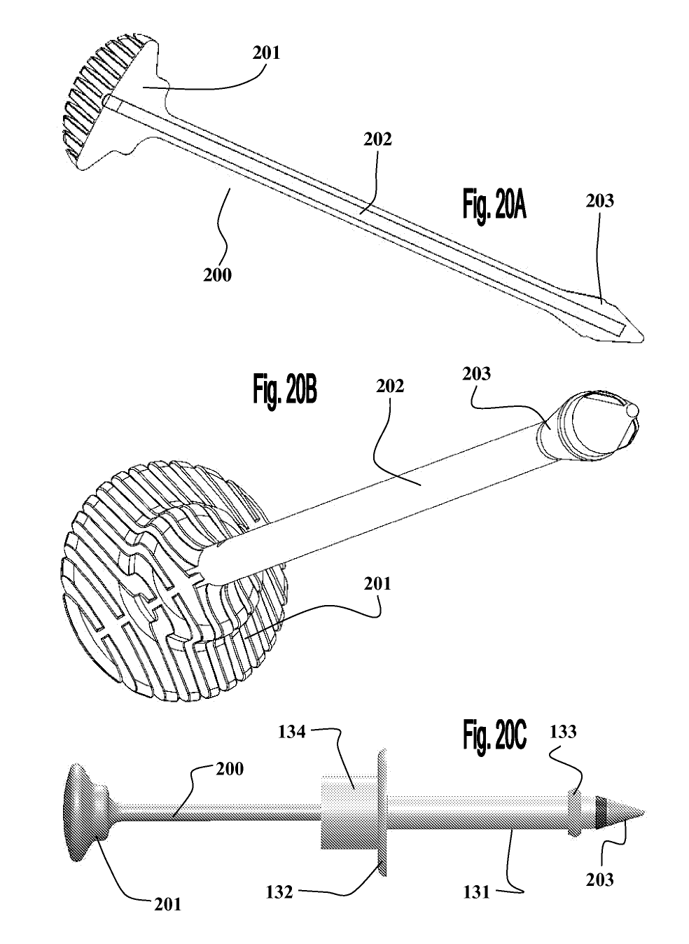

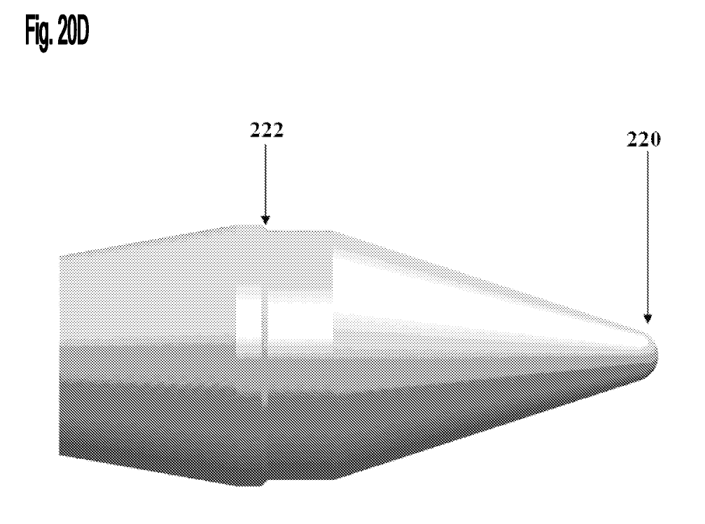

A final component part of the port is hollow distal tip 142 which is shown in FIG. 14 and, in longitudinal section, in FIG. 16. The purpose of this element is to provide an internal interface surface upon which a step structure located on the external surface of the trocar of the present invention (described hereinbelow and illustrated in FIG. 20D). By means of the interaction between the external step on the trocar and the internal step in the internal lumen of the hollow tip, said trocar is prevented from moving distally in relation to the port more than a distance that is predetermined by distance between said external step and the distal extremity of said trocar. A further purpose of the trocar-port step interaction is that it prevents the buckling or folding of the flexible port upon its trocar-driven insertion through the body wall. The aforementioned internal step in the port distal tip may be seen in the longitudinal section and enlarged view provided in FIG. 16. The hollow distal tip of the port of the present invention may be constructed of a rigid plastic such as (but not limited to) Ultem or polycarbonate, or a biocompatible metal such as stainless steel.

As explained hereinabove, the port of the present invention is flexible and may be constructed of silicone or any similar flexible biocompatible material (such as PPR rubber or any other medical rubber). The flexibility of the device is advantageous since it both permits the laparoscopic instruments inserted therein to be more accurately positioned within the body cavity. In addition, the flexible nature of the port permits its use in conjunction with curved instruments. The port may be constructed in any size desired. In one typical version, the diameter of the port is such that it is suitable for use with a trocar having an external diameter of 10 mm.

As mentioned hereinabove, in order to insert the above-described port through a surgical incision into a body cavity, it is desirable to use an appropriately-sized trocar to carry said port to its desired working position. In a preferred embodiment of the present invention a blunt-ended trocar containing an external step just proximal to its distal end is provided. An example of a suitable trocar 200 is shown in FIGS. 20A and 20B, in which said trocar may be seen to consist of an elongate shaft 202 ending proximally in a dome-shaped handle 201 and ending proximally in a conical tip 203. Preferably, the trocar is constructed from a rigid polymer such as polycarbonate or Ultem, or from a biocompatible metal such as stainless steel. In one preferred embodiment, the inner portion of shaft 202 may be reinforced with a metal rod (e.g. stainless steel 316, 304 or 302) in order to impart extra rigidity to the trocar. In some preferred embodiments (such as that shown in FIG. 20A), the trocar distal tip 203 may optionally be fitted with two or more wing-like structure to improve the penetrability of the trocar and associated port into the body cavity.

In one preferred embodiment, the trocar has an external shaft diameter of 10 mm. However, said trocar may also be constructed with a variety of different diameters without deviating from the scope of the invention. As mentioned hereinabove, the trocar of the present invention comprises an external step close to the widest part of its conical distal tip. This step 222, which is shown in the enlarged view of the trocar tip 220 shown in FIG. 20D, generally has a depth in the range of 0.02 to 1 mm. In one preferred embodiment, the step depth is 0.23 mm.

FIG. 20C illustrates the trocar 200 already inserted in its working position within the hollow shaft 131 of the dedicated port. The dome-shaped proximal end 201 of the trocar is sized and shaped to fit conveniently into the operator's palm.

In one embodiment the parts of the device, which extend outside the body when in use are removable, in order to improve the operator's visualization of the operative site and/or to prevent interference between the proximal portions of a plurality of instruments.

In another embodiment, portions of the device that are situated inside the body cavity in use may be selectively dismantled and removed following fixation and/or retraction of the desired organ or tissue, in order to prevent potential interference between instruments and ports within said cavity.

In one preferred embodiment, the various units of the device are either entirely disposable, or alternatively may be constructed as a combination of some disposable elements and other multi-use elements.

The device of the present invention and is various component parts may be sterilized by any conventional sterilization technique as well known to all skilled artisans in this field.

Use of the Present Device in Abdominal Surgery:

The surgeon may create an incision in the range of, for example, 0.5 to 15 mm in the abdominal wall of the patient and use this incision in order to insert the device (in its contracted state) into the body cavity. The device is then opened and maneuvered in order to create a discrete workspace (the surgical window), by means of displacing any tissues or organs (such as intestinal loops) that may otherwise obscure the surgical field. At this stage the device of the invention remains connected to the insertion port in a way that will allow the surgeon flexibility with operation and control of the device, device positioning, and anchoring (i.e. the ability to control and hold the device in the required position inside the body).

In another preferred embodiment, the device is inserted through a dedicated port as disclosed and described herein.

In yet another preferred embodiment, the device is inserted through a standard port as used in conjunction with other laparoscopic instruments.

In another preferred embodiment the device inserted through the incision previously created for insertion of a laparoscopic port and will be placed beside it with special sealing plate. This conformation is schematically illustrated in FIG. 9, in which can be seen an incision, into which have been inserted a standard laparoscopic port 92, one side of which is connected to a sealing plate 96, through which a retraction device of the present device 93 is inserted. The function of said sealing plate is ensure a gas-tight seal between the port 92 and the body wall 95, in the region in which the device 93 is inserted.

At the end of the procedure, the surgeon will close the arms by rotating the dynamic cylinder in the control unit (or by any other control means present). The device will move into its contracted (closed) conformation, and then be removed from the body of the patient through the incision.

The above-described embodiments of the invention have the following unique advantages:
The device structure is rigid enough to withstand the aforementioned forces and pressures applied by the surrounding tissues at the surgical site and by the abdominal walls, while also possessing non-traumatizing soft, elastic edges and extremities.
The attachment to the treatment area (facilitated by the optional attachment plate) and the retraction of surrounding tissue is optimized.
The physical connection between the device and the insertion port results in improved control of positioning and anchoring of the device—i.e. the device may be manipulated through any desired angle and plane from outside of the body cavity.
The physical connection between the device and the insertion port may save an additional operator's hand, i.e. the device is self-retaining.
It is possible to use a plurality of retraction devices of the present in invention in a single procedure, allowing optimal retraction of surrounding tissues and better exposure of the surgical field.
Injury and trauma of surrounding tissues is minimized.
The device may be constructed as a low cost disposable device.

It is to be noted that while the preceding description has made repeated reference to the use of the presently disclosed and claimed device in laparoscopic procedures that are performed in the abdominal cavity, the use of said device is not limited to that anatomical site alone. Rather, the device of the present invention (in particular the retractor unit) may be used in any situation wherein there is a need for the endoscopic or laparoscopic insertion of a retraction device.

The above examples and description have of course been provided only for the purpose of illustration, and are not intended to limit the invention in any way. As will be appreciated by the skilled person, the invention can be carried out in a great variety of ways, employing more than one technique from those described above, all without exceeding the scope of the invention.

1. A surgical retractor suitable for laparoscopic insertion, comprising
an elongate shaft having two or more arms at its distal end, and a mechanism for controlling the mutual said arms located at its proximal end,
wherein a membrane is attached to said two or more arms, such that upon mutual separation of said arms, forms a non-planar surface suitable for use as retracting or holding tissues or organs.

2. The surgical retractor according to claim 1, wherein each of the distal arms are curved in one or more planes.

3. The surgical retractor according to claim 1, wherein said retractor comprises no more than two arms at its distal end.

4. The surgical retractor according to claim 3, wherein the elongate shaft comprises a hollow tube through which pass two connecting elements, each of which connects one of the distal arms with the proximally-located mechanism for controlling the mutual separation of said distal arms.

5. The surgical retractor according to claim 4, wherein the proximally-located mechanism for controlling the mutual separation of said distal arms comprises a pair of scissor handles, wherein one connecting element is attached to a fixed scissor handle, and wherein the second connecting element is fixed to a movable scissor handle, wherein said movable handle is capable of being caused to move between a plurality of defined positions, and is further capable of becoming immobilized into each of said defined positions.

6. The surgical retractor according to claim 1, wherein the membrane is a polymeric mesh.

7. The surgical retractor according to claim 6, wherein the polymeric mesh is formed from polyethylene.

8. The surgical retractor according to claim 1, wherein the elongate shaft is enclosed by an expandable sleeve.

9. The surgical retractor according to claim 8, wherein the expandable sleeve is a silicone sleeve.

10. The surgical retractor according to claim 1, further comprising means capable of anchoring said retractor to an immobile structure.

11. The surgical retractor according to claim 10, wherein the anchoring means comprise one or more straps attached at their medial ends to the proximal part of said retractor, and wherein each of said straps further comprises a clip attached to the free lateral end thereof.

12. A flexible laparoscopic port suitable for use in inserting a surgical retractor according to any one of the above claims into a body cavity, wherein said port comprises:
a flexible, elongate hollow tube suitable for passage of a surgical instrument therethrough;
an upper housing surrounding the proximal end of said tube, wherein said housing comprises one or more gas-tight seals;
a retention structure surrounding a portion of the distal part of said tube; and
a hollow distal tip contained within the distal end of said tube, wherein said tip comprises a circumferential step within its internal lumen.

13. The flexible port according to claim 12, wherein the upper housing comprises:
an upper seal comprising having a central aperture that is capable of expanding in diameter in order to provide a gas-tight seal around an instrument inserted into the lumen of said port; and
a lower seal capable of preventing the passage of air or other gaseous through the lumen when there is no instrument inserted into the lumen of said port.

14. The flexible port according to claim 12, wherein the upper housing comprises a seal capable of preventing the passage of air or other gaseous through the lumen when there is no instrument inserted into the lumen of said port, and
wherein a seal comprising having a central aperture that is capable of expanding in diameter in order to provide a gas-tight seal around an instrument inserted into the lumen of said port is provided in the lumen of the distal portion of said port.

15. The flexible port according to claim 12, wherein the retention structure is a flexible flange.

16. A trocar suitable for use in conjunction with flexible port defined in claim 12, wherein said trocar is characterized by comprising an external circumferential step located close to the distal end of said trocar, and wherein the size and shape of said step is such that it is capable of docking with the internal circumferential step of said flexible port.

17. A kit comprising:
a surgical retractor according to claim 1,
a flexible port according to claim 12; and
a trocar according to claim 16.

18. A method for retracting tissues, organs or other structures in a laparoscopic or minimally-invasive surgical procedure comprising the steps of:
a) introducing a laparoscopic retractor into the body cavity containing said tissues, organs or other structures,
wherein said retractor comprises an elongate shaft having two or more arms at its distal end, a mechanism for controlling the mutual separation of said arms located at its proximal end, and a membrane attached to said two or more arms, and
wherein the distal arms are in mutual contact when said retractor is introduced into said body cavity;
b) operating said mechanism for controlling the mutual separation of said distal arms, such that said arms separate from each other by a desired amount, thereby causing said membrane to form a non-planar surface that is suitable in shape for retracting said tissues, organs or other structures;
c) manipulating the proximal end of said elongate shaft in order to cause said non-lanar membrane surface to move or retain said tissues, organs or other structures; and
d) optionally anchoring said retractor to one or more structure outside of the patient's body by means of one or more straps connected to the elongate shaft, each of which are fitted with a clip for attaching to said structure(s).

19. The method according to claim 18, wherein step (a) comprises inserting the retractor through a laparoscopic port that has been previously inserted through the body wall into the body cavity being treated.

20. The method according to claim 18, wherein the surgical retractor further comprises a dual layer sheath enclosing the elongate shaft, and wherein step (a) comprises inserting said retractor directly through a surgical incision, following which said sheath is inflated by means of introducing and expansion fluid thereinto.
